(19) United States
(12) Patent Application Publication
(10) Pub. No.: US 2024/0335433 A1
(43) Pub. Date: Oct. 10, 2024

(54) CELL CYCLE SYNCHRONIZATION PROCESS

(71) Applicant: Dennis W. Stacey, Novelty, OH (US)

(72) Inventor: Dennis W. Stacey, Novelty, OH (US)

(21) Appl. No.: 18/627,617

(22) Filed: Apr. 5, 2024

Related U.S. Application Data
(60) Provisional application No. 63/457,433, filed on Apr. 6, 2023.

(51) Int. Cl. A61K 31/4375; A61K 9/00; A61P 35/00

(52) U.S. Cl. CPC A61K 31/4375; A61K 9/0019; A61P 35/00

(57) ABSTRACT

A normal cell cycle synchronization process includes repeated administration of a drug specifically toxic for a single cell cycle phase to selectively synchronize normal cells. The selective synchronization of normal cells allows for the selective targeting of abnormal cells with cell cycle phase specific toxins. Such selective targeting may be useful in the treatment of a number of medical conditions.

The present application claims the priority benefit of U.S. Provisional Patent Application Ser. No. 63/457,433, filed Apr. 6, 2023, the disclosure of which is incorporated herein by reference.

### BACKGROUND

The present disclosure relates to processes and compositions for synchronizing cells in normal tissues and to treatment therapies which may be administered after cell synchronization.

Cancer treatment has achieved remarkable outcomes considering the fact that many treatments are based upon empirical drug identification, or the targeting of molecules required for the growth of both normal and many tumor cells. For cancer therapy to move to the next level, it must be based upon a fundamental difference between the growth of normal and tumor cells.

The most fundamental characteristic of cell growth is the manner by which a cell passes through successive phases of the cell cycle. For a cell to divide it must first grow in size and mass, termed the G1 phase (Gap 1 phase). The cell then is ready to duplicate its DNA, termed S phase (Synthesis phase). Between S phase and mitosis (M phase) there is another period termed G2 phase (Gap 2 phase). The growth properties of a cell are defined by the length of each of these cell cycle phases, and the time between mitosis and entry into G1 phase of the next cell cycle. As reported previously by others, the proliferative cells in the crypt of the gut move from mitosis to the next G1 phase without delay, and are therefore continuously dividing. Previous studies performed by the inventor, however, revealed an unexpected characteristic of normal gut epithelial cell growth: a remarkably high degree of uniformity in cell cycle length. Thus, almost every growing cell in mouse gut displayed the same cell cycle length. This characteristic of normal cells was found to be altered in naturally occurring gut tumors, which displayed average cell cycle lengths different from normal cells, and wide variations in the length of the cell cycle from one cell to another.

The inventor of the present application previously found that the average length of the cell cycle in normal mouse gut tissues is near 14 hours. Some of the inventor's previous work is described in U.S. Patent Publication No. 2021/0275562 A1, which is incorporated by reference herein in its entirety.

### BRIEF DESCRIPTION

Disclosed, in some embodiments, is a process for synchronizing cells within normal tissues. The process includes administering a plurality of doses of a drug which is selectively toxic for a single cell cycle phase. Drugs may include camptothecin, a camptothecin analogue, a pharmaceutically acceptable salt thereof, and EDU or related compounds able to be incorporated into DNA during its synthesis. Each successive dose is administered from about 9 hours and 45 minutes to about 10 hours and 15 minutes after the previous dose.

The camptothecin analogue may be topotecan or irinotecan.

5-Ethynyl-2′-deoxyuridine (EdU) induction of synchrony may include any of a number of similar molecules with substitutions at the number 5 position of thymine. Mutations in the attached sugar in thymidine, however, are often not appropriate.

In some embodiments, the plurality of doses includes at least 4, at least 6, or at least 7 doses.

In some embodiments, 3 to 5 doses of drug may be administered, followed by a waiting period (e.g., 40, 50 or more hours without drug); after which another 3 to 5 doses are administered. This process may continue indefinitely with lower concentrations of topotecan.

In some embodiments, each dose contains from about 0.1 μg/kg to about 1.5 μg/kg.

In other embodiments, each dose contains 0.3 μg/kg to about 3 μg/kg.

The total amount of topotecan administered may be at most about 100 μg/kg in a one-week period.

In some embodiments, the administration is via injections.

The process may not result in the synchronization of tumor cells, or other abnormally proliferating cells in a tissue.

Disclosed, in other embodiments, is a process for treating small intestine cancer. The process includes administering a plurality of doses of camptothecin, a camptothecin analogue, a pharmaceutically acceptable salt thereof, or another cell cycle specific toxin. Each successive dose is administered from about 9 hours and 45 minutes to about 10 hours and 15 minutes after the previous dose.

Administration may be via local injection.

In some embodiments even lower doses might be utilized and the treatment repeated over an extended period of time. The dose would vary according to the number of repeated treatments to ensure the same total drug dosage is delivered over time. For example, it has been found that a 2-fold lowering of the dose allows the animal to avoid toxicity for up to 20 or more cycles of treatment.

In some embodiments the treatment scheme might be altered. For example, a series of 3-5 treatments every approximately 10 hours would be followed by a resting period of 40-60 hours; after which another 3-5 treatments would be performed. This cycle might be repeated. Brief treatments with increased dosages followed by rest periods have been shown to result in cell cycle synchronization with reduced toxicity to the mice.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following is a brief description of the drawing, which is presented for the purposes of illustrating the exemplary embodiments disclosed herein and not for the purposes of limiting the same.

FIG. 1 is a flow chart illustrating a non-limiting embodiment of a cell cycle synchronization method in accordance with some embodiments of the present disclosure.

FIG. 2 is a flow chart illustrating a non-limiting embodiment of a treatment method in accordance with some embodiments of the present disclosure.

FIG. 3 is a fluorescence photograph of mouse gut section. The basement membrane is at the bottom, with the crypt adjacent to the basement membrane, and the villi pointing upward. Red stain: cells incorporating BRDU injected just prior to fixation. Green stain: cyclin D1. The labeled cells indicate the crypt region of the tissue. It is these crypt cells whose labeling is studied throughout the descriptions to follow (FIGS. 6-11).

FIG. 4 is a graph illustrating Length of the crypt cell cycle. This is the summary of three experiments where BRDU injection preceded an EDU injection by the length of time listed. The percentage of cells labeled with both BRDU and EDU are listed, with the error bars indicating the standard deviation between the three separate experiments.

FIG. 5 is a fluorescence photograph of a naturally occurring gut tumor. BRDU (stained red) was introduced 14 hours previous to EDU (green). DNA is stained blue with DAPI. Notice that there is little overlap between the BRDU and EDU-stained cells, indicating the cell cycle time is much different than the 14 hr normally observed with crypt cells of the gut in the same animal.

FIG. 6 illustrates the appearance of unlabeled gut tissue. An injection of EDU was given 1 hr prior to fixation and staining of the tissue. Above are paraffin thin sections stained as indicated. The crypt region is that area labelled with EDU and containing cyclin D1. Cells of the villi extend downward and to the left.

FIG. 7 illustrates synchronization of crypt cell cycle by repeated topotecan injection. Two mice received 9 injections of topotecan, separated by 10 hours. EDU was injected into separate animals either 5 hours or 10 hours after the final topotecan injection. It is expected that at the time of topotecan injection, or 10 hours later, none of the crypt cells would be in S phase, and therefore susceptible to killing by topotecan. In this animal, very little EDU labeling is seen 10 hours after the final topotecan injection. The staining seen is primarily in the interstitial cells of the villi. At 5 hours after topotecan injection, however, it is expected the cells would move in mass into S phase, and would therefore be efficiently labeled with EDU, as is shown.

FIG. 8 includes photographs of staining for cyclin D1 following synchronization. Two animals were treated with topotecan 9 times, separated by 10 hours. As in previous studies, EDU was injected into separate animals 5 hours or 10 hours after the final topotecan injection. In this case, however, animals were stained for both EDU and cyclin D1. It is clear that while there were few cells labelled with EDU 10 hours after the final topotecan injection, a high proportion of the cells were nevertheless healthy and actively cycling as indicated by their staining with cyclin D1.

FIG. 9 illustrates the timing of cell cycle synchrony: 5 animals received 9 injections of topotecan every 10 hours. Then, at the times indicated, separate animals were injected with EDU. Tissues were collected 50 minutes after the EDU injection and stained for DNA, EDU and cyclin D1. At 2 and 10 hours following the final topotecan injection the crypt cells were in G1-G2 phases, and therefore not synthesizing DNA. At 4 hours, 6 hours and 8 hours following topotecan injection, on the other hand, the cells had entered into S phase and were actively synthesizing DNA as indicated by high levels of EDU incorporation. Significantly, in those cells not actively incorporating EDU (2 hours and 10 hours), a high proportion of cells contained cyclin D1, indicating their active progression through the cell cycle in phases other than S phase.

FIG. 10 illustrates that tumor cells are not synchronized by topotecan. Four mice received an injection of Lewis lung carcinoma cells. After the tumors had developed the animals received 9 topotecan injections separated by 10 hours. At 1 hour, 5 hours, and 10 hours following the final topotecan injection, EDU was injected, and tissues collected 50 minutes later. As a control, a tumor-bearing animal without topotecan injection was also given EDU and tissues collected. Images are presented of EDU stains of the gut crypt region and of the tumor. A DNA stain of the tumor is also presented. Cells in the crypt region were actively labeled EDU only in the animal given EDU 5 hours after the final topotecan injection as previously seen, indicating that the crypt cells have been synchronized by the topotecan injections. On the other hand, the labeling of the tumor was indistinguishable between the animals, indicating the tumor had not been synchronized in the cell cycle, despite the fact normal cells had.

FIG. 11 illustrates injections spaced apart by times other than 10 hours do not synchronized crypt cells: Nine Injections of topotecan at the same concentration as in all other experiments were performed into 5 mice, but in this case the injections were spaced apart by 9 hours rather than the usual 10 hours. EDU was injected at the indicated times following the final topotecan injection. In this case, however, there was no evidence of cell cycle synchrony since EDU was not actively incorporated into crypt cells at any time following the final topotecan injection. Synchronization obviously requires injections spaced apart by 10 hours.

FIG. 12 is a graph illustrating the inhibition of Lewis lung carcinoma growth following treatments with topotecan and EDU. Tumor cells were injected on Day 1, while drug treatment started on Day 3. The size of tumors at various times is listed above, along with the drugs the animals were treated with (see FIG. 13). Cell Cycle Therapy involving topotecan alone, EDU alone, or a combination of the two was highly efficient in blocking tumor growth.

FIG. 13A-C illustrates a treatment schedule of animals shown in FIG. 12. Animals were treated every 10 hours, 40 minutes. FIG. 13A gives results for animals receiving only EDU injections, FIG. 13B for mixed injections, and FIG. 13C gives results for animals injected only with topotecan. Each box represents one treatment cycle. The number of each treatment cycle is listed above the figures, together with an indication of the number of days of treatment. The treatments are indicated by the colored box. Light boxes with a red outline indicate cycles without treatment; blue boxes-topotecan; and black boxes EDU. The color of the small boxes above the treatment row indicates the weight loss of the animals, with the color indicating how much weight was lost. The wide blue lines at the top indicate the tumor size in untreated (PBS injected) animals for comparison. The smaller line immediately above the treatment row indicates the size of the tumor in the treated animal. The top half of each figure shows results for the first 25 treatments, and the bottom half treatments 26-49. Notice that except for EDU alone, tumors in the treated animals appeared long after both control animals had to be euthanized due to tumor size.

FIG. 14A-C illustrates some aspects of a preclinical efficacy study in mice. Lewis lung carcinoma cells were placed under the skin of 24 mice. Three days later they were divided into three equal groups and injections began. The Cell Cycle group received three injections of topotecan 10 hours, 40 minutes (one cell cycle) apart; followed by a rest period of 53 hours, 20 minutes (five cell cycle periods); and the sequence was repeated. The Daily group received one injection of topotecan every 24 hours. All injections were the same in both sets (0.2 ml injections, 0.2 ml of 0.18 mg/mI topotecan). The Control group received injections of PBS at the same time as the Cell Cycle group. The experiment continued 9.5 days after drug treatments began. FIG. 14A: It is apparent that percent weight loss, the indicator of overall toxicity, was dramatically reduced by injections separated by the length of the cell cycle, compared to the daily injections; even though the same amount of drug was introduced throughout the experiment. For comparison, the control group slightly increased in weight. FIG. 14B: Daily injections were perhaps slightly more efficient in blocking tumor growth compared to injections separated by the length of the cell cycle (although the difference was slight). Both topotecan treatments induced dramatic decreases in tumor growth compared to the control group. (Actual tumor size is approximately 50% of the volume listed in the figure). FIG. 14C displays the sequence of topotecan injections in the Cell Cycle group compared to the Daily group. Notice that the same number of injections take place in the 15-day period displayed; only the timing between injections varies. This particular experiment continued until each group had received exactly 9 injections. (It has been found that injections separated by the length of the cell cycle are more tolerable when interspersed with rest periods equal to multiples of the cell cycle length.)

FIG. 15 is a graph illustrating that the combination of topotecan and EDU blocks tumor growth. Lewis lung carcinoma cells were implanted 4 days before treatment with topotecan (Cell Cycle Inj), or phosphate buffered saline (Control). All treatments were performed every 10 hours, 40 minutes. Tumors appeared 4 days thereafter. At 13 days of treatment, the topotecan-treated animals were divided into three groups (two animals in each group), and treatment with EDU was initiated. Three treatment protocols were tested. In the first, EDU was given three times followed by one treatment with topotecan. In the second group, two treatments of EDU were followed by two with topotecan. In the final group, one treatment with EDU was followed with three treatments of topotecan. After these treatments, the animals remained untreated for 5 treatment cycles (53 hours, 20 minutes), and the entire cycle was repeated three times (10 days). Average tumor sizes for each group are reported. It is clear that the treatment schedule with one EDU and three topotecan injections followed by 5 rest periods actually blocked or dramatically reduced the rate of tumor growth compared even to the effective inhibition with topotecan alone (three topotecan injections followed by 5 rest periods as described in FIG. 14C).

### DETAILED DESCRIPTION

The present disclosure may be understood more readily by reference to the following detailed description of desired embodiments included therein and the drawings. In the following specification and the claims which follow, reference will be made to a number of terms which shall be defined to have the following meanings.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent can be used in practice or testing of the present disclosure. All patent applications, patents, and other references mentioned herein are incorporated by reference in their entirety. The materials, methods, and articles disclosed herein are illustrative only and not intended to be limiting.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases that require the presence of the named ingredients/steps and permit the presence of other ingredients/steps. However, such description should be construed as also describing compositions, mixtures, or processes as “consisting of” and “consisting essentially of” the enumerated ingredients/steps, which allows the presence of only the named ingredients/steps, along with any impurities that might result therefrom, and excludes other ingredients/steps.

Unless indicated to the contrary, the numerical values in the specification should be understood to include numerical values which are the same when reduced to the same number of significant figures and numerical values which differ from the stated value by less than the experimental error of the conventional measurement technique of the type used to determine the particular value.

All ranges disclosed herein are inclusive of the recited endpoint and independently combinable (for example, the range of “from 2 to 10” is inclusive of the endpoints, 2 and 10, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

References to dosages in “μg/kg” refer to μg of active ingredient per kg bodyweight of the patient being treated.

The term “cell cycle” is not in any way limited to the measured cell cycle length of a tissue under any specific condition. As will be shown hereafter, the length of the cell cycle varies under different conditions and following different treatments. The cell cycle length refers to the length between mitotic events for any tissue under any particular circumstances. In practice, it is the length of time between individual cell cycle specific drug administrations that results in reduced toxicity, so long as it approximates the known length of the cell cycle under those conditions.

The methods of the present disclosure relate to cell cycle synchronization methods and their practical application. After normal cells are synchronized, the applications may include treatments which target specific cell cycle phases at particular intervals to selectively target abnormal (e.g., cancer) cells.

In order to utilize differences in cell cycle length as a target to selectively eliminate tumor cells, a strategy termed Cell Cycle Therapy is described here. For this approach, it is first necessary to identify a toxin that is highly selective for one and only one phase of the cell cycle. In addition, this toxin must be short-lived in circulation, so that there is little time for a cell to pass between cell cycle stages following its introduction. Such a toxin would be added multiple times to a tissue, with the time between separate treatments equal to the length of the cell cycle of normal cells. These normal cells may be gut crypt cells, or any other normal cell whose protection is critical in cancer therapy. In all known cases, these properties would differ from a tumor.

Illnesses, abnormalities, and tumors can result from cells in the body that develop altered growth characteristics. While it is common to consider growth abnormalities primarily in the context of tumors, there are many problems of proliferation; including, psoriasis, dermatoses, atherosclerosis, wound healing, scarring, maintenance of tissues such as the pancreas leading to diabetes, aging, and many other adverse situations. These diseases often result from mutations or other disruptions of normal growth control pathways, resulting in altered progression through the cell division cycle. To address these illnesses, powerful pharmaceuticals able to selectively target molecules involved in growth regulation have been developed. The results have been notable; but in many cases fall short of a cure because the targeted molecules play similar roles in both the normal and abnormal cells.

The processes of the present application may target a tissue with any abnormal growth condition other than cancer. In this case, the length of time between drug treatments would be the length of the cell cycle of the tissue to be preserved. It must be different than the tissue to be abolished. In the case of skin abnormalities, treatments might be topical.

The potential of cell cycle synchronization, and the elimination of abnormal cells that are not synchronized because of different growth characteristics, extends to considerations of aging. As tissues age, growth properties of the stem cells undoubtedly change. There is every reason to believe, however, that while many stem cells in a tissue might grow more slowly, there is still a population of these cells that retain near normal growth properties. It is further likely that these more normal stem cells would retain growth properties similar to young stem cells, and different than those altered by age. A protocol similar to Cell Cycle Therapy might eliminate stem cells with growth properties altered by age and allow their replacement with the stem cells more similar to those present in a younger tissue. The protocol to be used to repopulate a tissue with robust stem cells would be analogous to that described here for tumor treatment. Such a protocol should, therefore, be covered by the general concept outlined in this application.

To understand this strategy, an analogy may be helpful. Compare the cell cycle to a pie sliced into 4 parts. Each cell cycle phase is represented by a specific slice of this pie, designated here by position on the clock. A person sits and scoops out equal-sized slices of the pie periodically, representing the action of a toxic drug. This person, however, is able to reach pie and take scoops only from the 3:00 o'clock position (analogous to a drug that is toxic only during one stage of the cell cycle). The pie is placed on a turn table that rotates as the person eats (representing the constant progression of cells through the cell cycle). The speed of the turn table represents the length of the cell cycle. If the turn table rotates once every 5 minutes and the person takes a scoop exactly every 5 minutes, the vacancy left by the first scoop will be precisely located at the 3:00 o'clock position for the next scoop. The person will scoop into the cavity left by the first scoop and will obtain no more pie. In fact, even after many 5-minute scoops, no more pie will be obtained. The pie will remain whole, with the exception of the single first scoop. However, if the pie is placed on a turn table that rotates every 6 minutes (or any time other than every 5 minutes), the vacancy left by the first scoop will not be in exactly the 3:00 o'clock position when the person takes the next scoop 5 minutes later. The person will obtain more pie at every successive scoop until presumably, over time, the entire pie is consumed.

As indicated by this analogy, successive administrations of the cell cycle specific drug are spaced apart by exactly the length of the uniform normal cell cycle. Cells in the targeted cell cycle phase will be killed by the first treatment. The first scoop is successful in all cases. If a second administration of the drug takes place after exactly the length of one cell cycle, all the cells will be in exactly the same cell cycle phase as they had been at the time of the first treatment. Thus, the only cells able to be killed by the second treatment would be those that had already been killed by the first treatment. Cells in all other cell cycle phases will consistently be protected, in the same way the bulk of the pie on the 5-minute turn table remained untouched. This scheme would require that all cells have highly similar cell cycle lengths, as this laboratory (and this laboratory alone) observed for normal gut epithelial cells. Cells with altered growth characteristics, however, with either altered cell cycle length or with a wide variation in cell cycle length, would be like the pie on the 6-minute turn table. At each drug administration there will be abnormal cells in the targeted cell cycle phase available for killing. Over time, all abnormal cells could conceivably be killed, while most of the normal cells would be spared.

Eukaryotic cells pass through a cell cycle including two gap phases (G1 and G2), a synthesis phase (S) during which genetic material is duplicated, and an M phase in which nuclear division (mitosis) is followed by cell division (cytokinesis). The phases in sequence are G1→S→G2→M. During the G1 phase, the cellular content except for the chromosomes is duplicated. Next in the S phase, the chromosomes are duplicated by the cell. Subsequently in the G2 phase, the cell checks the duplicated chromosomes for errors and makes any necessary repairs. Nuclear and cell division occur in the M phase. A tissue growing with a uniform cell cycle length will normally have cells in all cell cycle phases at any given time. The time from one mitosis to the next, however, is restricted within a narrow range. On the other hand, a tissue with cell cycle synchrony would not only have cells with similar cell cycle lengths, but at any given time, all the growing cells of the tissue would be in the same cell cycle phase. The cells would then move in mass from one cell cycle phase to the next, such that if all cells of the tissue were in G1 phase at a given time, they would then move in mass into S phase, then to G2 phase and so forth. This would never occur naturally in most adult tissues. The induction of cell cycle synchrony in gut cells has never been observed before these studies. The induction of synchrony discussed in this document would be considered highly unexpected by most investigators. Its induction by cell cycle specific drugs is a validation of the anti-cancer treatment model described in this document.

It is emphasized that while cell cycle synchronization of the gut might be highly useful in the achievement of selective tumor killing, it has other potential uses. For example, the synchronization of a normal tissue would allow the analysis of growth control in that tissue in ways previously impossible. Thus, a biochemical description of the events leading to passage through each step in the cell cycle could be accomplished by simple biochemical analysis of the entire tissue at specific times. Much effort has been invested in such studies in cultured cells in the past, but the resulting synchrony is more the result of cells being released from quiescence of cell cycle blockers, rather than simple passage through the cell cycle. Moreover, studies in culture can only approximate the results of studies in a normal tissue. Such studies are made almost trivial by the tools described in these studies.

In addition, cancer is only one of many abnormalities resulting from uncontrolled cell growth. Use of this synchronization procedure may well prove valuable in the treatment of a plethora of other abnormalities, such as psoriasis, aging, scaring, and others.

This invention will allow investigators the study and to manipulate growing tissue cells in ways not previously imaginable. Prompted by this discovery, studies in research laboratories will very possibly lead to clinical studies, and advances in health care in many areas. Of critical importance is the potential that this might well represent a paradigm shift in cancer treatment. When cancer, which is a disease of altered growth, is treated based upon this altered growth, outcomes heretofore unachievable are likely to result.

The S phase may be of particular interest in accordance with some methods of the present disclosure. Accurate duplication of the genetic material is critical to cell division. The methods of the present disclosure take advantage of the degree of uniformity in cell cycle length of some normal cells (e.g., small intestine cells) by specifically targeting S phase cells in treatment steps as discussed in more detail below.

In alternative embodiments, M phase cells may be similarly targeted using a drug that selectively kills mitotic cells (e.g., vinblastine and its derivatives). The M phase-targeting drug may block the formation of the mitotic spindle. It may bind tubulin and block the completion of mitosis.

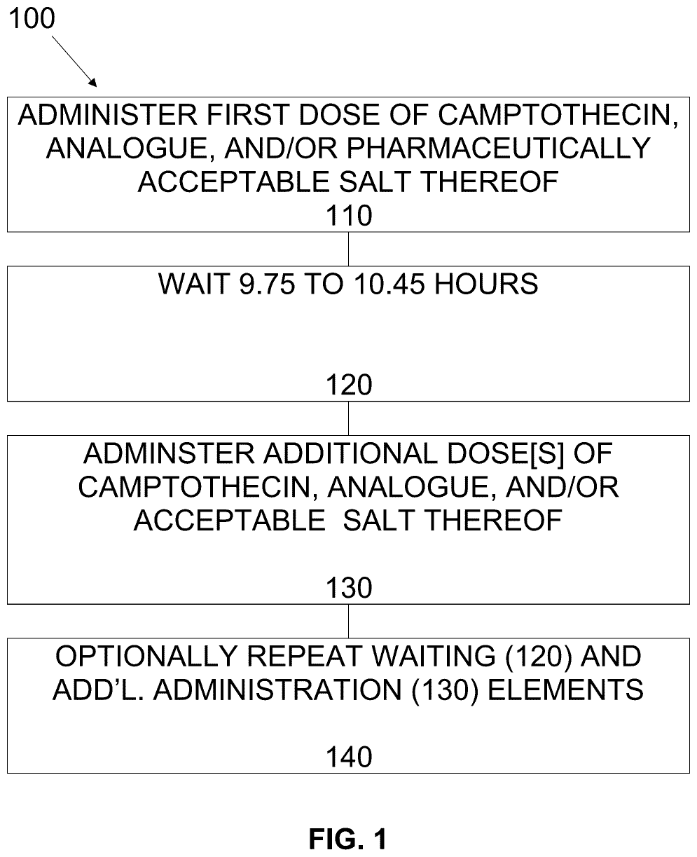

FIG. 1 is a flow chart illustrating a non-limiting embodiment of a cell cycle synchronization method. The method 100 includes administering a first dose of camptothecin or an analogue or pharmaceutically acceptable salt thereof 110, waiting 9.75 to 10.25 hours 120, administering an additional dose of camptothecin or an analogue or pharmaceutically acceptable salt thereof 130, and optionally repeating the waiting and additional administration elements 140. The method drives normal cells into synchrony, allowing them to thrive in the continued presence of the cell cycle specific toxin. Abnormal cells, without the growth characteristics of normal cells, are not synchronized; thus, the cells are eliminated.

Non-limiting examples of camptothecin analogues include topotecan and irinotecan.

In addition to camptothecin and related constructs, S phase specific toxicity and cell cycle synchronization have also been observed with high concentrations of EDU itself. Studies in the laboratory of the applicant have recently demonstrated that EDU does not interfere with cell cycle progression in the first cell cycle phase after its administration, but is toxic to cells in the next cell cycle following its incorporation. On the other hand, camptothecin and related drugs are immediately inhibitory to cell cycle progression, to the extent that they block or delay passage through the upcoming mitotic cycle. EDU and the topoisomerase II inhibitor etoposide have been shown, along with camptothecin and topotecan (topoisomerase I inhibitors), to be selectively toxic to cells in S phase. Only topotecan and high concentrations of EDU, however, have been tested and shown to induce cell cycle synchrony in the mouse gut. These two reagents have entirely distinct mechanisms of action. Their only similarity is highly selective S phase toxicity. The ability to induct cell cycle synchrony in mouse gut is most likely, therefore, due to their toxicity in a single cell cycle phase. It is anticipated that any drug with such cell cycle selective toxicity will also be able to induce synchrony, although this must yet be shown for drugs toxic in phases other than S phase.

Because the three S phase specific toxins identified to date have completely separate mechanisms of action, it is expected that other drugs will also be identified to have toxicity for one cell cycle phase compared to another. There is no fundamental reason to favor a drug selectively toxic for one cell cycle phase compared to another, although it might well be easier to identify drugs selectively toxic for S phase, and perhaps for M phase, compared to G1 or G2 phases. For a drug to function in this protocol, it is necessary only that it have a much greater toxicity for one cell cycle phase than another. In addition, it must be removed from circulation within the length of the targeted cell cycle phase to avoid large numbers of cells moving between phases while it remains toxic. All drugs with this mechanism of action are contemplated by the present disclosure.

The total number of doses may be in a range from about 4 to as many as 20 or more. Longer treatments, however, would most likely involve periods of rest from drug treatment. These periods would likely be multiples of the normal length between doses, such that the length of the cell cycle would be considered in all cases, whether or not a dose of drug is administered during a given cell cycle phase.

For longer treatment protocols, 4 consecutive does are described below, with a wait between 4-dose regimens of 20 to 70 hours (4-7 cell cycle lengths). With this protocol, prolonged drug treatments may be appropriate for maximal killing of abnormally cycling cells. Different dosing strategies, however, are also possible, and may prove useful in appropriate circumstances.

Each dose in the experiments described here were from 0.5 to 1.5 μg/kg of the active ingredient; and the total cumulative dose administered may be at most 50 μg/kg; although variations in dosage may improve on the outcome depending on the tissue, species, drug, and application.

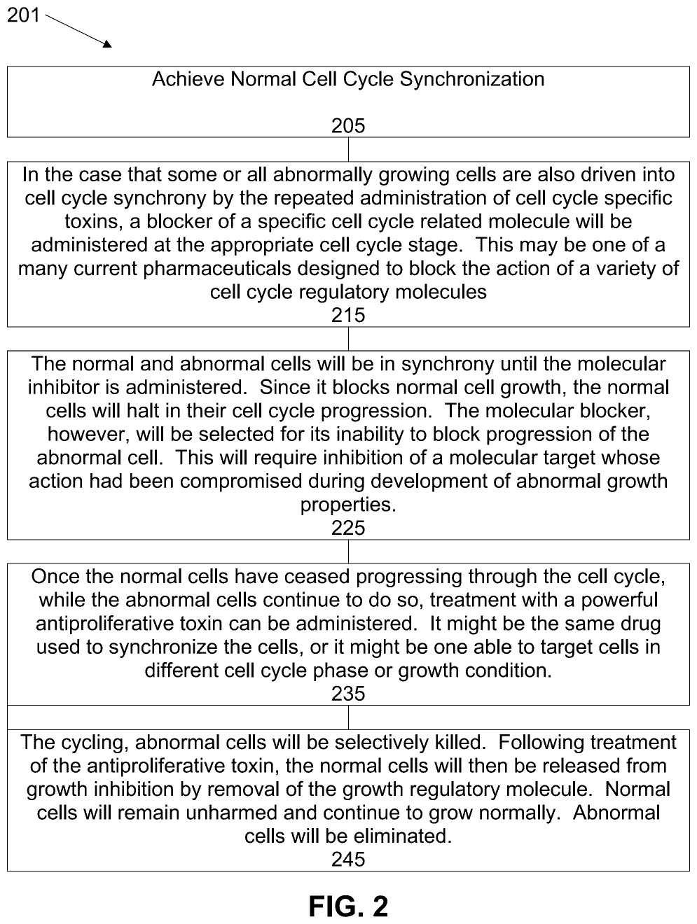

FIG. 2 is a flow chart illustrating a non-limiting embodiment of a secondary treatment method 201. Treatment with an S phase specific toxin has been shown to synchronize normal mouse gut cells. At the same time, such treatments do not synchronize Lewis Lung carcinoma tumor cells in a mouse. This is presumably due to the difference in growth characteristics between normal and the tumor cells. Moreover, there are many types of gut tumors that develop in APC-deficient mice. Most of these have been shown in the applicant's laboratory to grow with cell cycle phases totally distinct from normal gut cells. It is, therefore, expected that most if not all tumors and other abnormally growing cells will fail to be synchronized by the treatments described above. Without synchronization, the abnormal cells will of necessity be killed. However, it is possible that in a rare instance abnormally growing cell could exhibit growth characteristics similar to normal cells or that a population of these cells with similar growth characteristics to normal cells might be selected, such that the abnormal cells themselves might become synchronized in the cell cycle by the treatment. This would allow the abnormal cells to escape the toxicity described above. Should this unusual situation arise, the abnormally growing cells would be rendered highly sensitive to a two-phase treatment option described in FIG. 2.

The method becomes appropriate only in the unexpected instance where the abnormally proliferating cell type is also synchronized, along with normal cells, by repeated administration of the cell cycle specific toxin. Should this take place, selective toxicity of abnormally proliferating cells could be achieved following treatment with an inhibitor of a normal growth promoting molecule, such as Ras, a cyclin dependent kinase, or a cyclin. For example, synchrony would be induced 205 as usual by repeated administration of the cell cycle specific toxic as in FIG. 1. Should this treatment also synchronize some or all of the tumor cells, a growth inhibitory drug would be used to block progression of normal but not abnormal cells 215. This drug would target one of the normal regulators of cell cycle progression. It would not be toxic, but would have the ability to block cell cycle progression temporarily, but only in normal cells. It would obviously need to target one of the cell growth regulatory molecules mutated or otherwise altered in the tumor. Blockage of cell cycle progression in normal but not abnormal cells will result following treatment with an inhibitor of a cell cycle regulatory molecule, such as Ras, cyclins, or cyclin dependent kinases, or similar regulatory molecules (many of which are currently available). Such inhibitors are of use in this instance because they target molecules often altered in activity or expression in cells with altered growth properties. The particular inhibitor used will be chosen based upon its ability to block normal but not abnormally growing cells. Because of the large number of drugs targeting a wide variety of growth regulatory molecules, and because abnormal growth must of necessity involve the altered activity of at least a few normal growth regulatory molecules, there is every reason to believe that a currently available pharmaceutical can be identified with the ability to block normal but not abnormal cell growth in most if not every instance 225.

With abnormal cells continuing to progress through the cell cycle, and normal cells blocked in cell cycle progression, a toxin for growing cells will be administered. It will kill any cell able to proliferate, but may be chosen to target the specific cell cycle phase the synchronized abnormal cell is in. This final toxin will kill the tumor cells which continue to proliferate in the presence of the growth blocker. It will not harm the normal cells whose proliferation is temporarily halted 235. Finally, all treatments will be removed so that the normal cells can continue normal growth following the killing of abnormal cells 245.

Should tumor cells become synchronized in the cell cycle following Cell Cycle Therapy, the above scheme might be used to selectively kill those cells. This approach relies upon the fact that tumor cells have mutations in normal growth control molecules. Blocking the action of one of these molecules will inhibit the proliferation of normal cells, but not the tumor cells. Tumor cells synchronized in the cell cycle continue growing after such blockage, and are thereby sensitized to an antiproliferative drug. At the same time, the proliferation of normal cells is inhibited and the cells protected.

In particular embodiments, the treatment method is used to treat cancer of the small intestine.

In other embodiments the cancer to be killed would be of a type other than a gut tumor.

In other embodiments, a non-cancerous but harmful group of tissue cells will be killed based upon their growth characteristics which are altered compared to normal.

In order to utilize differences in cell cycle length as a target to eliminate abnormally growing cells, it is first necessary to identify a toxin that is highly selective for one and only one phase of the cell cycle. This toxin has to be short-lived in circulation, so its effects would be limited to only one phase of the cell cycle. Such a toxin would be added multiple times to a tissue, with the time between separate treatments equal to the length of the cell cycle for cells that are to be preserved, presumably normal cells.

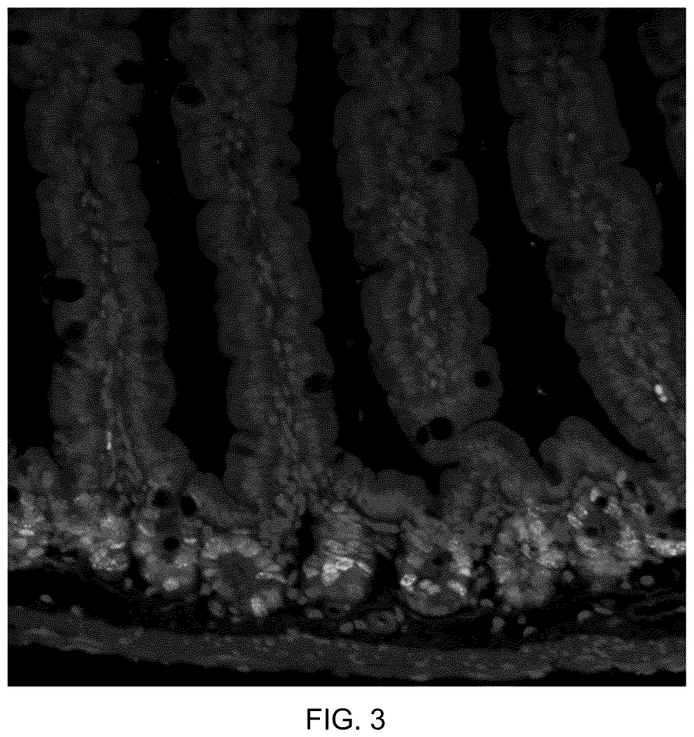

All animal studies described here were performed in the dividing cells of the gut, which are located near the basement and termed the crypt. Stem cells in the crypt region proliferate continuously and rapidly. Cell division in this limited region displaces cells upward towards the villi (FIG. 3) (Clevers and Batlle, 2013). As growing cells are displaced away from the basement membrane they stop proliferating, differentiate into one of four different cell types, and take their place in the villi. Cells sloughed off of the villi are replaced by the constant replication of stem cells in the crypt region. Because of their continuous replication, cells of the intestinal crypt are particularly sensitive to traditional cancer therapies, which often target growing cells. Gut toxicity is particularly difficult for patients who often struggle to obtain sufficient nutrients. Consequently, gut toxicity often limits the amount of chemotherapeutic drug tolerated, and therefore the overall level of tumor killing. A means to protect gut cells would, therefore, increase the efficiency of tumor killing in many cases.

Tumors result from mutations or other disruptions of normal growth control pathways, resulting in uncontrolled progression through the cell division cycle. To eradicate tumor cells, powerful pharmaceuticals able to selectively target molecules involved in growth regulation have been developed. These have been found by mass screening, selective targeting of selected growth control molecules, enhancement of antibody surveillance, alteration of the supportive environment, and other strategies. The results have changed the lives of many individuals; but at times fall short of a cure, because the targeted molecules are important for both normal and tumor cells, and cannot be totally eliminated. The only thing one might imagine to be completely different between normal tissues, and those exhibiting abnormal growth properties would be the growth properties themselves. If an anti-tumor strategy could be based entirely upon these growth differences, tumor elimination might be much more selective and efficient. Such a strategy, however, would rely upon the identification of a targetable difference in the growth properties of normal compared to tumorous tissues.

The invention described here is based upon original work performed by the inventor which identified for the first time such a critical difference in the growth characteristics of normal and tumor cells of the gut. Normal cells grow with a high degree of uniformity and tumor cells do not. The identification of this difference, together with means to exploit it in the selective killing of tumor cells, is the basis for the discovery described here. The result is that tumors may be killed with a strategy that spares gut cells, and therefore reduces or eliminates one of the primary limitations in cancer therapy.

Identification of the fundamental difference in growth between normal and tumor cells was achieved with the use of two stainable thymidine analogues; BRDU (Bromodeoxyuridine), and EDU (5-ethynyl 2′-deoxyuridine). These two molecules become stably incorporated into DNA during DNA replication (S phase). When either is injected into mice, it labels at high levels only those tissue cells actively replicating DNA (in S phase) at that time. Because these drugs are both rapidly removed from circulation, only cells actively replicating DNA within 1 hour of injection are labelled. If the second drug is introduced into the same animal at a later time, thereafter, it too will label only those cells in S phase at the time of the second injection. The tissues are then collected and stained for BRDU and EDU with separate fluorescence tags. The intensity of each marker is then determined by fluorescent image analysis. Fortunately, it is the characteristic of dividing cells that these two labels are incorporated most efficiently into nuclei of cells near the middle of S phase. When the two injections are separated by the length of the cell cycle, therefore, both markers will be stained at maximal levels in the same cell. The cell cycle characteristics of a tissue can then be determined by comparing results following injections separated by increasing lengths of time.

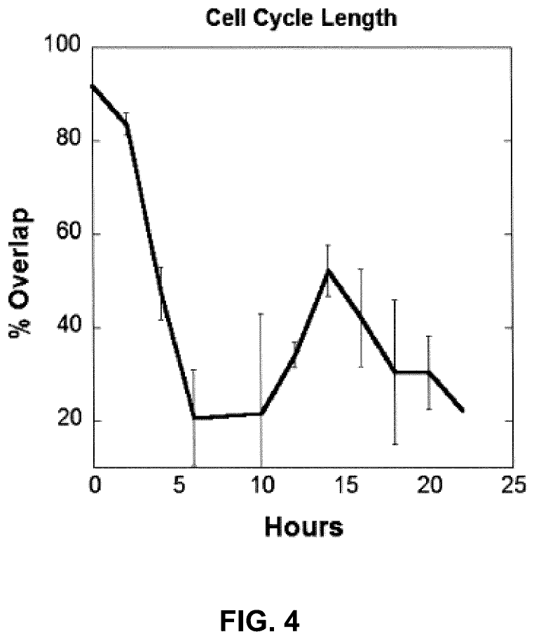

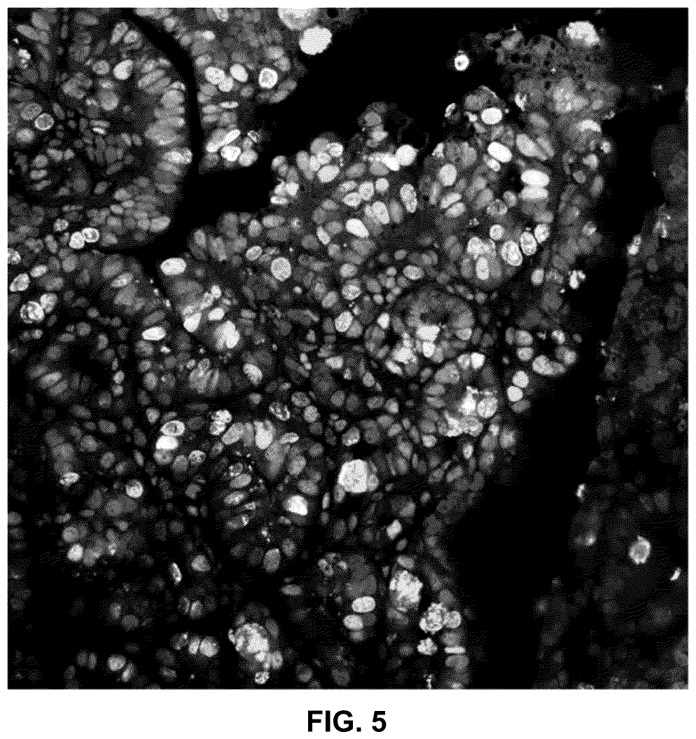

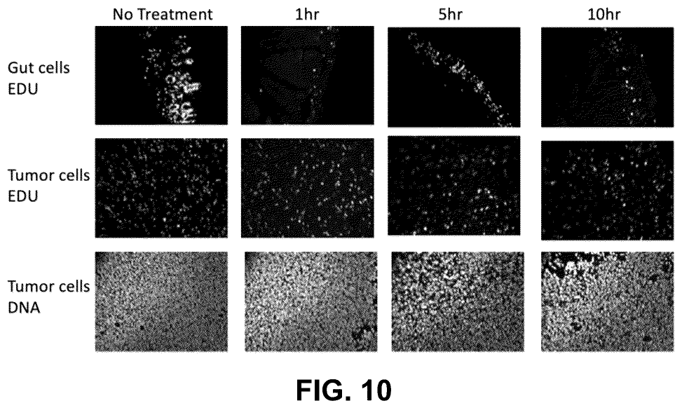

For the analysis performed by the inventor, BRDU was introduced into a number of mice at the same time. EDU was then introduced into individual mice at various times thereafter, from 1 hour to 20 hours. The results of several analyses were combined (FIG. 4). It was clear that the length of the average cell cycle in mouse gut crypt cells is near 14 hours. Surprisingly, and critically, it was also clear that there was a remarkably high uniformity between the cell cycle lengths of individual cells within the crypt (FIG. 4). Accordingly, the profile of co-staining of the two markers exhibited a sharp peak at 14 hours, with much lower numbers at longer and shorter times. For comparison, spontaneous tumors forming in the Min mouse model were studied. These mice lack one copy of the APC gene, resulting in the formation of numerous gut tumors between 3 and 5 months of age. Neither the average cell cycle length in these tumors, nor uniformity of cell cycle length, was observed in these tumors (FIG. 5). Similar results were observed with the Lewis Lung Carcinoma tumors to be described below (FIG. 10). These observations, which demonstrate conclusively for the first time the difference in growth properties of normal and tumor tissues, are the conceptual basis for the invention reported here. This information on cell cycle length has not been reported by others, and has been considered confidential information by this lab in the past.

It may be worth noting that the identification of uniform cell cycle length in crypt cells is not the first notable observation first made by the inventor and his laboratory. In the mid-1980s, the inventor first described the role of the RAS protein in a signal transduction pathway currently known as the MAP kinase pathway. This observation predated confirmatory observations in other laboratories by several years; and is currently the foundation of all studies of cell cycle regulation (Smith, Degudicibus and Stacey, 1986). This study was conducted with the technique of single cell microinjection which itself was invented by the inventor as a graduate student (Stacey and Allfrey, 1976). More recently, the inventor identified the molecular events that control passage through the cell cycle from G1-S phase transition to the decision to continue proliferating during G2 phase (Hitomi and Stacey, 2001) (Stacey, 2010). These observations once again required some time for confirmation by other laboratories, but are currently part of the central dogma of cell cycle control. It should not, therefore, be considered surprising that the inventor would identify a novel and critical new aspect of the growth characteristics of normal compared to tumor cells.

To exploit differences in growth properties as a means to selectively kill tumor cells, drugs were identified which were highly selectively toxic to only one phase of the cell cycle, and which were rapidly removed from tissues and from circulation. When these drugs were introduced into tumor bearing animals separated by the cell cycle length of normal cells (FIG. 1), these normal cells became synchronized in the cell cycle, and highly resistant to drug toxicity (to be described below). Tumor cells, however, were fully sensitive to the drug in the commonly used Lewis lung carcinoma model.

No approach anything like this one has ever been utilized or suggested before; because it is based upon unpublished information recently discovered for the first time by the inventor. No one can fully predict the future of an individual invention; but the progress of our society relies upon a constant flow of new ideas. It is clear from this application, and a review of the literature, that this invention is completely novel in every aspect. It may or may not be a watershed discovery, but it is an excellent example of the type of invention that has the potential to become such.

This invention includes the use of any cell cycle specific toxin or toxins applied according to the length of the cell cycle, even though the cell cycle may vary in length between different cells and under different conditions. It includes; 1) techniques to identify and test the cell cycle specificity of drugs, 2) means to determine the degree of synchronization of normal and tumor cells in the cell cycle, 3) the use of multiple cell cycle specific drugs together no matter the actual cell cycle phase targeted, 4) and the use of rest periods between drug treatments equal to the length of the cell cycle. It is not meant to cover drugs used for any treatment protocol unrelated to the cell cycle; but it does cover the use of any drug in a cell cycle specific manner.

The following examples are provided to illustrate the devices and methods of the present disclosure. The examples are merely illustrative and are not intended to limit the disclosure to the materials, conditions, or process parameters set forth therein.

### Examples

Identification of Drugs with Cell Cycle Specific Toxicity and a Brief Period of Toxicity.

The examples were performed on the contact inhibited cell line, NIH3T3. The growth characteristics of these cells are of paramount importance. They must be cultured so as to never reach confluence; since, like normal cells, they stop growing when becoming confluent on the culture plate. Thus, they are plated at 60,000 to 150,000 cells per 35 mm plate, and transferred to a new plate every 2-3 days. The cells must also be limited in passage number to ensure their growth rate is rapid. The definition of low passage cells will vary from one lab to another, and the actual cell cycle length of these cells may vary, but it is critical that from one experiment to another the length of the cell cycle remains constant. While the general characteristics of culture are critical, the identity of the cell line used is not. Any cell line whose proliferation is blocked by contact inhibition and/or reduction of the serum level in culture would be suitable for this application.

First test). Constant killing over a broad concentration range: The drug to be tested is diluted across a broad concentration range, and placed on separate actively proliferating NIH3T3 cultures for two hours. These cultures are then washed, passaged, and at a constant volume plated on a fresh 100 mm plate in 10% calf serum. This plate is left 6 days to allow individual living cells to form colonies. The plates are then fixed, stained with crystal violet, and the number of colonies counted and compared to a mock treated culture. The colonies indicate the number of living cells after drug treatment. If the drug is toxic only to a single cell cycle stage over a broad concentration range, there will be an extended plateau in killing. Thus, once a toxic concentration is reached, the same number of cells will be killed even if the concentration of the drug is increased up to a 100-fold greater concentration. This result indicates that once all the cells in the targeted cell cycle phase are killed, cells in other cell cycle phases will be killed only following a dramatic increase in drug concentration. It is critical, of course, that the drug be placed on the cells for a limited time, 1-3 hours, to ensure that large numbers of cells do not pass between cell cycle phases during drug treatment. Cloning of the cells following drug treatment allows a quantitative determination of the number of surviving cells.

It is also possible to perform this experiment in an exploratory way by treating sparsely growing cultures and determining how rapidly they reach confluence. If cells in all cell cycle phases are killed, the time to reach confluence will be increased, potentially resulting in complete elimination of viable cells in a narrow concentration range. Cloning of the cells described first removes the potential artifact resulting from simple slowing of cell growth by a drug.

Second test). Serum stimulated growth: NIH3T3 cells stop growing and enter quiescence when serum is reduced in the medium to 0.3%, so long as they are at a sufficient density on the plate. Thus, 120,000 cells were plated on a 35 mm plate and left 18 hours prior to serum deprivation. After 24-36 hours there are no mitotic figures apparent, and the cells are flat in appearance. When 10% serum-containing medium is added back to this serum-deprived culture, the cells withdraw from quiescence and enter the cell cycle. Extensive studies have shown that they enter G1 phase about 8 hours after serum addition, S phase at about 12 hours, and mitosis about 16-20 hours after serum addition. These times are presented as examples, and may vary between different cultures and cell types. Following serum stimulation, these cells are in similar cell cycle stages throughout the period of serum induction, but they are not synchronous in the truest sense, because they exhibit considerable difference in cell cycle length. The drug to be analyzed is added for 3 hours at various times following serum addition, and then washed off. At the end of the experiment the treated cultures and control cultures are cloned to identify the number of surviving cells, as described above. With this strategy it is possible to identify the actual cell cycle position of toxicity. Moreover, it allows the toxicity at one cell cycle position to be directly compared to toxicity at other cell cycle positions. The identification of S phase cells, for example, involves treatment of separate serum-stimulated cultures with the drug from 6-9 hours following serum addition (G1 phase) and from 13-16 hours following serum addition (S phase). For an S phase-specific toxin, G1 phase cells will not be harmed, and the S phase cells will be killed over a wide concentration range. Killing of cells in mitosis would involve treatment from 16-22 hours following serum addition. The precise times would be determined by pulsing the culture with EDU at the tested times, staining, and determining the actual number of S phase cells in each time period. Mitotic cells are identified by fixation and counting mitotic figures microscopically.

Third test). Phase specific toxicity: As described above, it is expected that any cell cycle specific toxin will have reduced toxicity if added multiple times with the length of the cell cycle separating separate treatments. For this experiment, the cultured cells are plated so as to be able to pass through at least 3 doublings prior to becoming crowded on the plate, which reduces the rate of cell cycle progression. It was found that only actively cycling cells can be utilized for this study. They are then plated at 30,000 cells per 35 mm plate, and the experiment started 10-15 hours later. One treatment with the drug takes place at this time. The drug is left 2 hours and washed off. At varying times, a second treatment and then a third treatment are added to the cultures and then removed 2 hours later. The time between treatments varies from plate to plate, but the same time is used for each individual plate. Thus, one plate is treated three times, once every 12 hours, another every 13 hours and so forth. Finally, the cells are passaged and a small portion of the cells plated on to a 100 mm plate for 6 days as a clonal analysis of the number of cells surviving the treatment. A cell cycle specific drug will show reduced killing when the time between treatments is equal to the length of the cell cycle (12 to 19 hours, but generally near 15 hours). In practice, timed additions to cultures in this way can result in up to 30% survival of cells under conditions where treatments at other times kill all the cells.

Each of the treatments described above are able to identify cell cycle specific toxicity during any cell cycle period. Only method #2 would allow identification of which cell cycle period is being targeted.

Three drugs were identified among those initially tested to be cell cycle specific. They happened all to be S phase specific. These drugs are:
EDU (ethynyl-2-deoxyuridine); which was toxic for only S phase cells from 10 micrograms/ml to 0.1 micrograms/ml.
Camptothecin and its soluble analogue topotecan, which were selectively toxic to S phase cells from 0.1 microgram/ml to 0.001 microgram/ml. Because of its solubility, high level of toxicity, and high degree of selectivity for S phase cells, most studies were performed with topotecan.
Etoposide, which was selectively toxic for S phase cells only over a 10-fold concentration range. Other drugs tested were not selectively toxic during S phase cells.

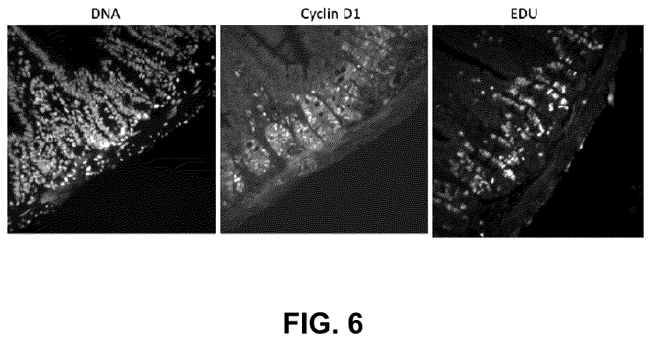

Test in living animals: Following the identification of drugs (topotecan, EDU) with the ability to kill S phase cells in culture with a high degree of selectivity, their activity in living animals was tested. All experiments were performed according to separate protocols approved by IACUC committees at the following institutions: the Northeast Ohio Medical University, and Case Western Reserve University. Studies of cell cycle length were approved by the IACUC of the Cleveland State University. Recall that the proliferating cells of the small intestine are located near the basement membrane in regions termed the crypt. Cells in this region are the only cells that proliferate in the gut, with the exception of rare proliferation of support cells in the matrix of the villi. Epithelial cells in the crypt region are essentially all proliferating all the time. Consequently, they are actively labeled by a pulse of EDU to label cells in S phase. Cells not labeled with EDU are in G2-G1 phases, and are labeled with cyclin D1 (FIG. 6).

Toxic concentrations of topotecan are well established, but it was first necessary to determine topotecan toxicity with 9 injections, one every 8-20 hours. Analyses began with approximately 10% of the LD50 concentration. It was then escalated until overall toxicity to the animals was observed. The final concentration of topotecan injected was 0.33 mg/ml-0.2 mg/ml; 0.05-0.27 ml per injection (depending on the mouse strain and weight of the mouse). Following determination of general topotecan toxicity following multiple injections, it was necessary to determine if, as predicted, injections spaced apart by the length of the cell cycle would be less harmful to the animals.

A total of 9 injections were performed for each time interval. These intervals were chosen according to the length of the gut epithelial cell cycle, which has been shown to be between 13 and 15 hours. The times first chosen were 8, 12, 14, 16 and 20 hours between injections. Thus, 9 injections at 8 hours, or 9 injections at 12 hours, 14 hours, 16 hours or 20 hours were performed, and the effect of these upon the animals determined. The 8 hour time was selected to definitely be shorter than the cell cycle, and 20 hours to be longer than the cell cycle, so that negative controls would be built into the protocol. After repeated experiments, however, there was no observable difference in the ability of animals to survive repeated injections at any of these times. In each case, the injected animals had to be euthanized due to loss of weight, hunched appearance, disheveled coat appearance, and lethargy. Rather than totally abandoning the study, however, tests were performed at different times, leading to a totally unexpected result. Animals injected every 10 hours showed essentially no adverse effects to the 9 repeated topotecan injections. Animals injected every 9 hours and every 11 hours, on the other hand, were negatively affected as were animals injected at any of the other times listed. It is considered totally unexpected, almost shocking, that the 10-hour animals were the only ones protected from toxicity following the repeated treatments. This indicates a totally unexpected degree of similarity in the length of the cell cycle in proliferating mouse gut cells.

The 10 hour timing of protective injections emphasizes one of the most important aspects of this invention. Cell cycle length of stem cells (crypt cells in this case) appear to be highly variable in different situations. This has been observed repeatedly in the experiments to be reported below. While all studies relied on a specific timing shown to be best tolerated by mice over an extended period of time, there are clear evidences that prolonged treatments with somewhat different times can also provide protection from drug toxicity and induce synchrony. In this case, the timing is related to the cell cycle characteristics of the particular stem cell in the particular conditions, rather than to a predetermined cell cycle length in resting animals. Moreover, cell cycle synchrony may be induced during an extended part of a treatment protocol, but from time to time may be lost without compromising the protective nature of protocol itself. This invention describes the use of appropriate cell cycle specific drugs in a timed sequence which is shown to reduce drug toxicity, and which relies on the cell cycle characteristics of the normal cells to be protected, and abnormal cells to be eliminated. These cell cycle characteristics are those exhibited under the conditions of treatment.

Additional studies indicate that there are sequences of 10-hour injections better tolerated than others, even when the same total drug is injected over time. Consecutive 10-hour injections were not tolerated by mice as well as when rest periods separated a sequence of 10-hour injections. The mice tolerated injections well if four or fewer injections were given consecutively, followed by a period of 20 to 60 hours without injection. Thus, four 10-hour injections, followed by rest periods of 20 hours, 30 hours, 40 hours, 50 hours 60 hours etc. were well tolerated. The amount of drug given during each injection was adjusted to ensure that the total drug over time did not vary between injection sequences. Mice tolerated 4 injections every 10 hours, followed by rest periods of 20-40 hours particularly well. Once again, the aspect of this invention covered in this application is the fact that drugs are repeatedly administered, interspersed by rest periods, each of which relate to multiples of the cell cycle length under treatment conditions,

### Cell Cycle Synchrony

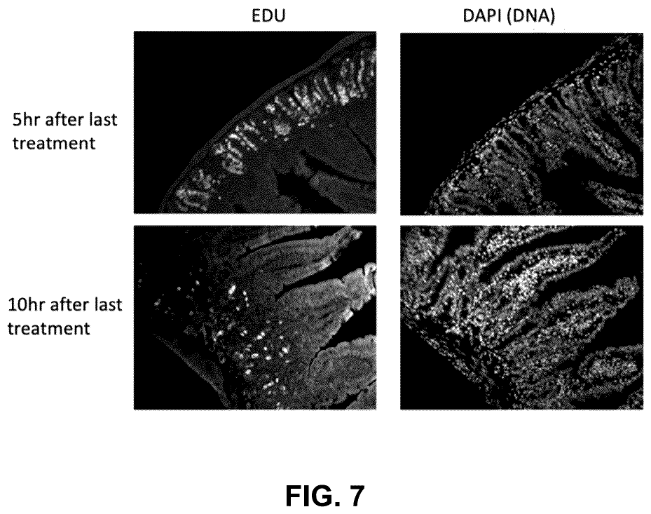

Although it might be expected that the gut cells in treated animals would become somewhat aligned in their cell cycle progression by the repeated topotecan injections, the results were well beyond expectation, almost shocking. All the epithelial cells in the gut tissues of animals injected 9 times every 10 hours were in essentially the same cell cycle phase at all times. The injections had driven the entire tissue into complete cell cycle synchrony. This was apparent when gut tissues were studied at various times following the final 10-hour injection. If, for example, the tissue was studied immediately following the final injection, no cells were in S phase as indicated by their inability to incorporate EDU, a DNA synthesis marker. However, if the animals were analyzed 5 hours following the final injection, essentially all the cells were in S phase (FIG. 7). Finally, the tissue was analyzed a full 10 hours following the final topotecan injection. This is the time at which the next successive topotecan treatment might have taken place. As expected, to ensure that the tissue would be protected from a potential next topotecan injection, none of the cells were in S phase at this time, as indicated by failure to incorporate EDU (FIG. 7).

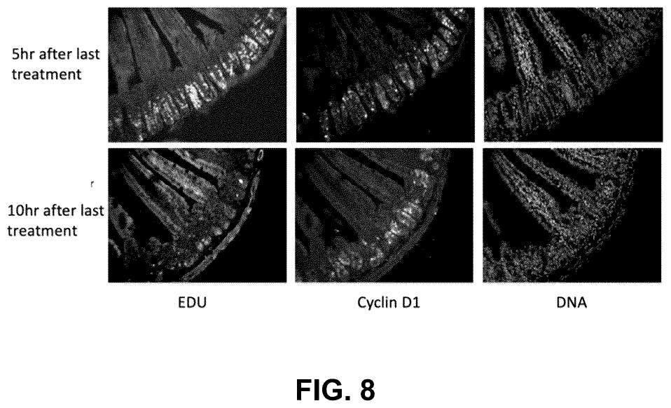

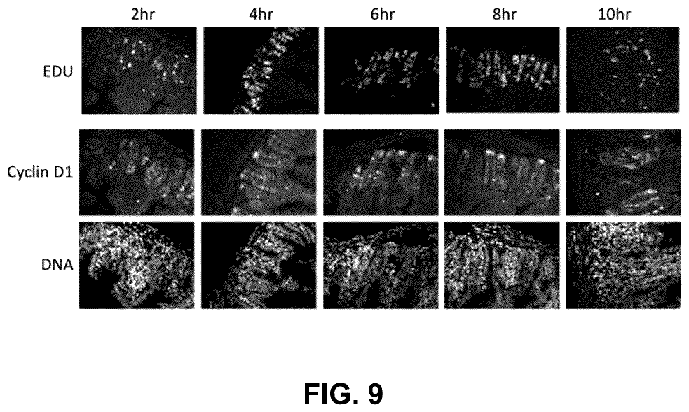

As a control to the above studies, the tissues described above were stained for the expression of cyclin D1. It was previously demonstrated that cyclin D1 is expressed throughout the cell cycle of actively proliferating cells, except during S phase. Thus, upon entry into S phase the level of cyclin D1 is always reduced to near zero. It returns to high levels of expression upon entry into G2 phase, but only in cells that are actively proliferating. Therefore, an actively proliferating cell would incorporate EDU if it were in S phase, and it would express cyclin D1 if it were in any other cell cycle phase. If all the cells of a tissue either incorporate EDU or express high cyclin D1 levels, all the cells of the tissue are actively proliferating. To demonstrate that the gut epithelial cells in animals following 10-hour topotecan injections were actively cycling, the tissues described above were stained for cyclin D1 expression in addition to EDU incorporation. Those cells in the crypt which failed to incorporate EDU expressed high cyclin D1 levels. Thus, immediately after the final topotecan injection, or 10 hours following this injection, the cells were generally negative for EDU incorporation. In these tissues, however, cyclin D1 was expressed at high levels. On the other hand, at 5 hours after the final topotecan injection, when the majority of the cells incorporated EDU, cyclin D1 expression was apparent in a greatly reduced proportion of the cells (FIG. 8). Thus, all cells continued to actively pass through the cell cycle in a synchronous fashion. They were either positive for EDU staining indicating they were in S phase, or for cyclin D1 staining, indicating they were in G1 or G2 phases (FIG. 8). Further analysis was performed at 2 hour intervals from 0 hours to 10 hours following the final topotecan injection. It is clear that cells between 4 hours and 8 hours following the final topotecan injection were in S phase, incorporated EDU, and were low for cyclin D1 expression. On the other hand, cells at 2 hours and 10 hours following the final topotecan injection they in G1 and G2 phases as indicated by low levels of EDU incorporation, and active expression of cyclin D1 (FIG. 9).

There are a very few EDU positive cells observed at 1 hour and 10 hours following the final topotecan injection. Careful analysis, however, indicates these are not the epithelial gut cells normally cycling in the crypt region of the gut. Rather, these are the few connective tissue cells in the space between epithelial cell layers (stromal cells). Normally, the actively cycling epithelial cells obscure the appearance of the few interstitial cells that take up EDU. The labeled stromal cells, however, become apparent in animals with highly synchronized epithelial cell populations (FIG. 7). The fact that they are not synchronized is evidence that only cells with the growth characteristics of the normal epithelial cells had been synchronized.

Sequential injection series: When 4 injections of topotecan every 10 hours were separated by 40 hours without injection, the total amount of drug tolerated by the animals increased. Thus, even though each injection contained a greater amount of drug, these were better tolerated when interspersed with rest periods. This regimen also induced a high level of cell cycle synchrony when sufficient topotecan was injected. Separate animals received injections of 0.20 ml of topotecan at 0.33 mg/ml, 0.20 mg/ml, or 0.12 ml of 0.20 mg/ml topotecan. Each animal was given four 10-hour injections of a given concentration, separated by 40 hours without injection; repeated four times. At 5 hours following the final topotecan injection, BRDU was injected into the animals to identify cells in S phase between the timed topotecan injection. Animals injected with BRDU were then injected again 5 hours later with EDU (10 hours after the final topotecan injection), to identify cells in S phase at the time the next successive topotecan injection would have taken place. 50 min following the EDU injection tissues were collected and analyzed. When injections of the two higher concentrations of topotecan were performed, synchrony was almost complete. With an injected concentration of only 0.12 mg/ml, however, very little if any synchrony was observed. In this as in other experiments, cell cycle synchrony was demonstrated with three separate analyses. A high proportion of BRDU positive cells indicated most cells were in S phase 5 hours following the final topotecan injection. A low number of EDU positive cells indicates that the cells have left S phase by 10 hours following topotecan. A high proportion of cyclin D1 positive cells would indicate that at the end of the experiment, 10 hours following the final topotecan injection, the cells are actively cycling and in G1/G2 phase (as in FIG. 9).

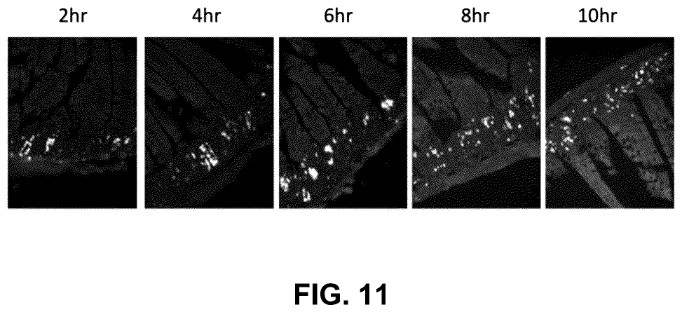

To demonstrate that injections spaced apart by 10 hours were the only ones able to induce synchrony, animals received 9 injections of topotecan spaced apart by other times, and their tissues were analyzed. In no case were the tissues synchronized by injections separated by times other than every 10 hours. Thus, animals receiving 9 injections separated by 9 hours displayed unhealthy gut morphology and few gut cells able to incorporate EDU at any time following the final injection (FIG. 11). Similar toxicity was observed following 9 injections separated by 11 hours. Even greater toxicity to the gut tissue was observed with injections spaced apart by 8 hours or 12 hours. Clearly, the cell cycle synchrony of gut cells is induced by repeated injections, but only within a narrow time window. This cell cycle synchrony was clearly correlated with the ability of the treated animals to tolerate the injections of topotecan. Repeated injections with cisplatin, a toxin which is not cell cycle specific, showed no decrease in toxicity regardless of the time between repeated injections; and at no time were the cisplatin injected cells synchronous in the cell cycle.

The fact that topotecan induces cell cycle synchrony only within a 1-hour time range (10 hours) is one of the most remarkable aspects of these observations. As indicated above, studies with cells in culture suggest that even in cultures synchronized by serum deprivation and stimulation, the cell cycle length varies broadly. There is no precedent in culture to predict the extremely demanding time restraints involved in synchronization of mouse gut cells. Indeed, such a limitation was considered so unlikely that the 10-hour time point was almost completely overlooked. This observation in animals receiving 12 injections might be contrasted with animals in the studies to be discussed below where injections continued for over 20 days. In these animals synchrony was at times less apparent than at earlier times, even though drug tolerance was similar. As noted above, more needs to be understood regarding prolonged cell cycle synchronization. The important observation is the fact that even in extended studies, the animals tolerated high drug levels and tumors were inhibited in growth (see below).

While the measured cell cycle length in mice of various strains was from 13-15 hours, repeated injections of a highly specific S phase toxin were well tolerated in these same mice only when introduced every 10 hours. If the injections were spaced apart by the expected cell cycle length, the animals were not protected. It is not clear why this might be the case. Presumably, the strong selective pressure applied by the cell cycle specific drugs had induced the cells to cycle more rapidly than normal.

### Lack of Synchrony in Tumor Cells

For the Cell Cycle Therapy to work, it is necessary that normal cells become synchronized in the cells cycle, and that tumor cells in the same animal do not. The failure of the repeated topotecan injections described above to synchronize tumor cells was performed in Lewis lung carcinoma tumors. All studies of tumor killing were performed exactly as described above for studies of cell cycle synchrony. Thus, 9 injections of topotecan were separated from one another by 10 hours in C57Black mice carrying a tumor of approximately 0.5 gm. EDU was injected into three different topotecan treated animals at 1 hour, 5 hour or 10 hours as a marker for DNA synthesis. A control tumor-bearing mouse received mock injections and was injected with EDU 5 hours after the final injection. Gut tissue together with normal gut tissue was collected and analyzed. FIG. 10 shows the labeling of gut tissue and of tumor tissue. The normal gut tissue at 5 hours has a majority of cells in S phase and therefore labeled with EDU. At 1 hour and 10 hours, however, these normal tissues in the tumor-bearing animals show very little labeling, except for the stromal cells as described above. A low level of labeling at 1 and 10 hours, compared to high labeling in between these times is a clear demonstration that the topotecan had induced cell cycle synchrony in the normal gut cells of these animals. The tumor cells, however, were not synchronized as indicated by a similar level of EDU labeling at all times.

Use of this Invention in Other Tissues

While the ability of gut tissues to be synchronized by repeated topotecan injections is contrasted with the tumor, it is highly possible that other tissues, particularly those with active proliferation, would also be synchronized by such treatments, although the times might be different. Thus, normal skin tissues might become synchronized by topical topotecan treatments, while any abnormally growing tissue in the skin might fail to be synchronized, and therefore killed. This might potentiate the elimination of any skin lesion with growth properties different than normal skin, even if it were not a tumor. Even skin regions with reduced proliferative capacity might be eliminated and replaced by healthy cells. The strategy to reduce tumors described in this document should be considered in the context that it can be directly applied to other pathologies in other normal tissues, particularly those with constant proliferation of stem cells. A person having ordinary skill in the art, after reviewing the present application, would be capable of optimizing dosages, times between treatments, etc.

### Psoriasis Treatment Protocol (Proposed)

An example of a protocol for the treatment of psoriasis is presented as an example only, and does not exclude other protocols based upon the principle of Cell Cycle Therapy. Psoriasis is a chronic autoimmune condition characterized by the rapid proliferation of skin cells, resulting in thick scales and red patches. Fortunately, there are excellent mouse models for the study of this condition (Gangwar, Gudjonsson and Ward, 2022). The effectiveness of any strategy will be compared to the use of topical imiquimod therapy. This commonly used approach modulates the immune response by targeting receptors on macrophages and related cells to induce apoptosis. Any cell cycle-based strategy would be compared to this. However, since Cell Cycle Therapy is based upon an entirely different approach, it might also well be used in conjunction with topical imiquimod treatment to improve overall outcome.

The mouse models initially selected for study would be the Asebia autosomal recessive mouse model; which results in alopecia, and thickened dermis. Alternatively, the Flaky skin mouse is an autosomal recessive mutation resulting in inflammation, and hyperkeratosis. As with psoriasis in general, these are believed to induce alterations in the growth of keratinocytes as the result of immunological abnormalities (Gangwar, Gudjonsson and Ward, 2022). It should be noted that numerous mouse models of psoriasis have been developed by genetic manipulation. These hold great potential in a study such as this, but will not be discussed further here.

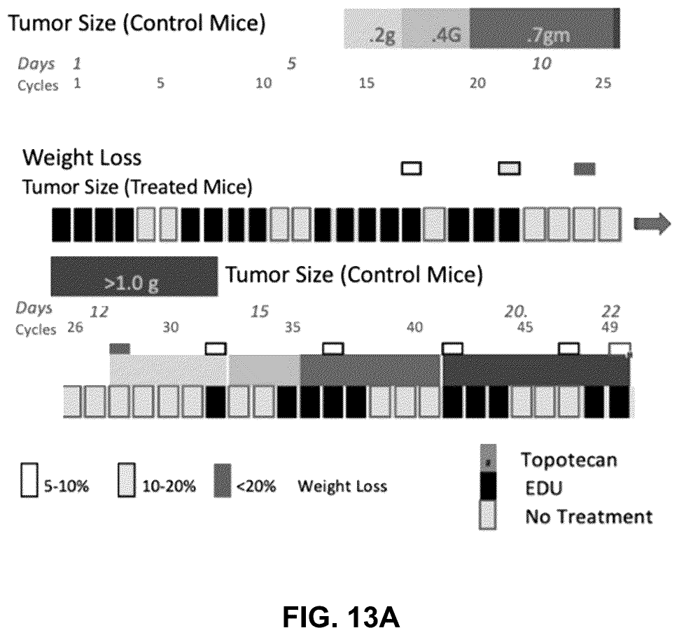

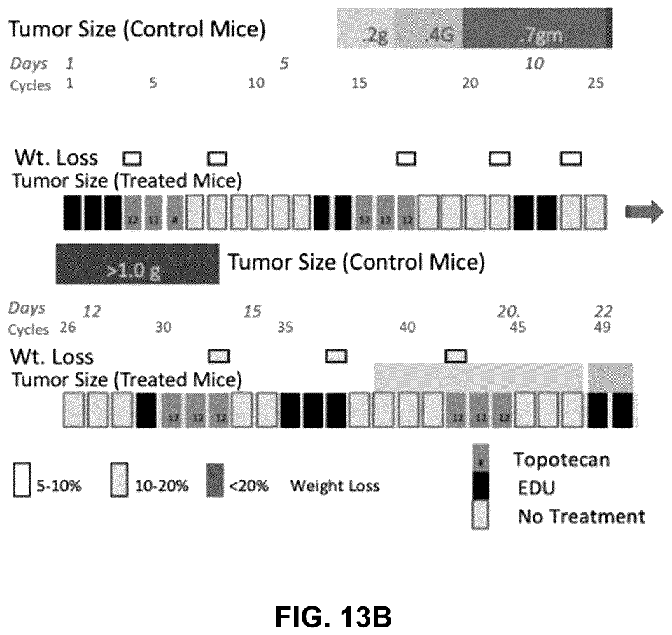

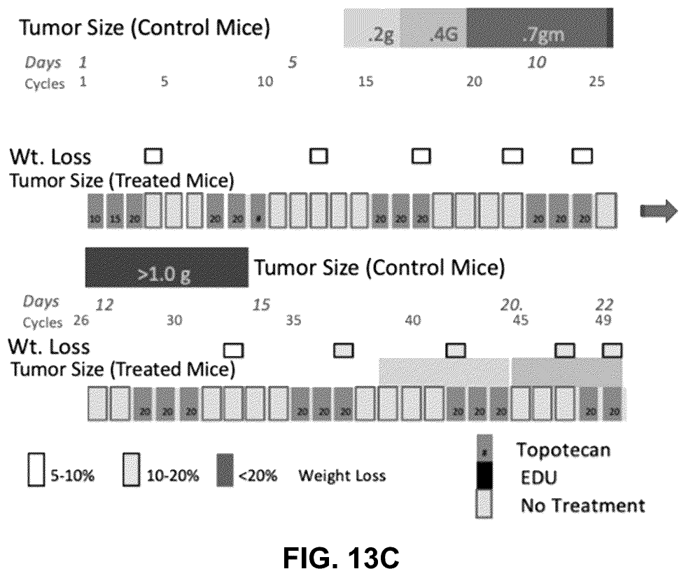

Experiments would begin with a topical preparation of topotecan (0.5 mg/ml) and EDU (10 mg/ml). A dose response experiment would determine tolerable concentrations to be administered every 10 hours 40 minutes. Administrations would mirror those described in FIGS. 13A-C and 14C. Drug toxicity would be indicated by increased irritation of the skin. Once the greatest tolerated dose is determined its effect upon skin inflammation and thickening would be determined both visually and histologically. A positive result would be indicated by the return of skin to normal thickness, the regrowth of hair, and reduced signs of irritation. Histological markers of reduced symptoms would involve loss of epidermal hyperplasia is seen by a reduced thickening of the epidermis and elongation of the rete ridges (called acanthosis). In addition, the normal thickness of all skin layers would be expected, together with the loss of unusual numbers of immune or inflammatory cells and structures. If even limited results are observed, further studies of skin cell synchronization in the cell cycle will be conducted.

A major concern for topical skin treatments is the requirement that any drug have a limited length of effectiveness, to ensure that movement of cells between cell cycle stages during treatment is limited. While a treatment can easily be removed from the skin, it would need to be determined how long the clearing of drug already within the skin takes. This could be determined by treatments with topical EDU preparations followed by removal and treatment with an BRDU preparation. The length of time required to observe cell populations labeled with EDU but not BRDU would indicate the length of time required to remove EDU from the tissue. Topotecan dramatically reduces the rate of DNA synthesis (as observed by the inventor). Topotecan treatments would be removed and followed at various times thereafter with an BRDU treatment. Removal of topotecan from circulation would be indicated by the return of normal rates of DNA synthesis, and therefore of BRDU incorporation. If rapid removal is not observed, other carriers of drug would be considered.

### Demonstration of Anti-Cancer Effects

The anti-tumor model proposed here relies upon the identification of drugs able to selectively target one stage of the cell cycle with high specificity, and to be rapidly removed following injection into an animal. Once topotecan and EDU were identified with these properties, they were tested with the Cell Cycle Therapy approach described above, to determine their ability to inhibit tumor growth. The results were highly confirmatory of the overall model.

Before anti-tumor studies were conducted, however, topotecan and EDU were injected in various schemes to determine their long-term toxicity, together with the ability to induce synchrony. All previous studies had been conducted in CD-1 mice from Charles River. As discussed above, topotecan was least toxic, and induced synchrony best, when injections were spaced apart by 10 hours. However, Lewis lung carcinoma cells are syngeneic only for C57Bl/6 mice (Charles River and other vendors). All tumor studies were therefore conducted in this mouse strain.

Extensive studies were conducted to determine the optimal spacing between injections for C57Bl/6 mice, as differences between mouse strains might exist. As discussed above, a series of 9 injections of topotecan were performed into C58Bl/6 mice. Injections were spaced apart by 9 hours 30 minutes, 9 hours 45 minutes, 10 hours, 10 hours 15 minutes, 10 hours 20 minutes, 10 hours 30 minutes, 10 hours 40 minutes, 11 hours and 11 hours 20 minutes. After the 9 injections spaced apart by these times were complete, the animals were weighed over the next 10 days to determine their overall health. It was observed that injections spaced apart by 10 hours 40 minutes were best tolerated by C57Bl/6 mice. This injection interval was, therefore, used in all studies involving Lewis lung carcinoma tumors in C576Bl/6 mice.

Clearly, an important aspect of this invention is the fact that spacing between injections will vary. For these reasons, it is essential to determine the optimal time between drug treatments with multiple injections. The interval inducing least toxicity while retaining toxicity to the tumor would be chosen. Assessment of synchrony would be supportive. As indicated above therefore, this invention covers any treatment involving repeated exposure to cell cycle specific toxins, whose toxicity is reduced by periodic introduction. It is assumed the length between introductions would be related to the length of the cell cycle; but it must be understood that this length will vary between individuals and conditions.

Furthermore, this invention also takes into consideration the dynamic nature of cell cycle under conditions of prolonged treatment with cell cycle specific drugs. This fact is particularly well illustrated by the fact that when injections of topotecan were performed every 24 hours for up to 20 days, cell cycle synchrony of the gut was observed and tumor killing was enhanced. In this case, prolonged drug treatment with topotecan had forced the gut cells into cell cycle synchrony outside the range considered to be otherwise optimal. In this case, the crypt cells divided every 12 hours, with two cell cycle between each injection. Tumor killing was enhanced by this protocol.

It appears that prolonged injections of a cell cycle specific toxin might drive cells into synchrony even when the times between individual injections vary somewhat. If the injections involve a cell cycle specific toxin, or multiple such drugs, and if cell cycle synchrony results from such treatments, they are covered by this invention. The only exception would be in the case of a pre-existing treatment protocol identified to be effective without knowledge of these studies, even if cell cycle synchrony is incidentally involved.

### Extension to Human Studies:

This leads to a question regarding the use of this invention in treatment of human illness. Clearly, the first step would be to determine the concentration and interval between injections which produce the least side effects. This study would be rather straight forward, but it would be valuable to follow it up with a determination of gut cell cycle synchrony. In studies of mice, synchrony always has involved injection of EDU and/or BRDU, which should be avoided in humans if at all possible. Fortunately, there is an excellent alternative, although it does still require a gut biopsy. It turns out that cyclin E is expressed exclusively during S phase of normal human cells, while cyclin D1 is expressed in all phases of a cycling cell except S phase. Therefore, a gut biopsy could be stained for cyclin E and cyclin D1. This test would be performed at various times following the final drug treatment, as described above for studies of mouse tissues. Depending upon the timing, a tissue expressing either a high proportion of cyclin E staining compared to cyclin D1 staining, or the opposite, would indicate cell cycle synchrony. If the biopsy were collected at the time of the next successive treatment, cyclin D1 should predominate. If the biopsy were performed at a time intermediate between treatments, cyclin E would predominate. This is the case if an S phase specific toxin were used. If a toxin specific for another cell cycle phase were used, the timing would differ accordingly.

### Determination of the Best Treatment Protocol in C57Bl/6 Mice:

Because anti-tumor studies of necessity must be conducted over an extended period of time, studies were conducted to determine the best regimen for extended treatments. Recall that all studies of synchrony were limited to 9 injections in less than 4 days. Anti-tumor studies, however, must extend over a longer period of time. It was found that for treatments of up to one month or longer, toxicity was reduced when rest periods were introduced between successive 10 hours 40 min injections. From 2 to 4 drug injections were best tolerated when from 2 to 8 rest periods (also of 10 h 40 min) separated them. In some cases, much longer rest period were tested, and found to be conducive to tumor killing. If toxicity were identified in any individual animal (loss of more than 15% body weight), rest periods were added until the animals returned to appropriate weights, at which time treatments were continued. Inhibition of tumor growth was not lost with these prolonged periods of rest. It is assumed that shorter rest periods be in multiples of 10 hours 40 min; while longer rest periods (many days) may be independent of cell cycle length prior to the reinitiation of the cell cycle-timed protocol.

The Lewis Lung Carcinoma model is a commonly used tumor model for assessment of general anti-tumor activity. The cells can be grown in culture and then introduced underneath the skin on the back of syngeneic C56Black mice. When 1-2 million cells are introduced, tumors begin to form within 6-10 days. This is an extremely aggressive tumor, with doubling times of 3 days or less. Generally, the effectiveness of a therapeutic treatment is measured in the delay of tumor growth, with delays on the order of a two-fold increase in doubling time considered positive (Van Moorsel et al., 1999). In rare occasions, with an extremely effective treatment, the time of tumor appearance is delayed for a week or more. In this study 2 million cells were injected underneath the skin on the mid back of the mice. The tumor was measured with calipers to determine its size. To confirm size measurements, the tumor was excised in total and weighed at the end of the experiment. All anti-tumor drug injections were intraperitoneal (IP). Mice in the range of 8-16 weeks of age were utilized, but within a given experiment, mice within the same age range were generally used for comparison. All studies were conducted under standard guidelines, and conformed to the protocol approved the Case Western Reserve and the Northeast Ohio Medical University IACUC. Weight loss was measured to determine animal health, with loss of over 20% an indication for experimental termination. Similarly, tumors were not allowed to grow beyond 15% of body weight.

### Anti-Tumor Studies:

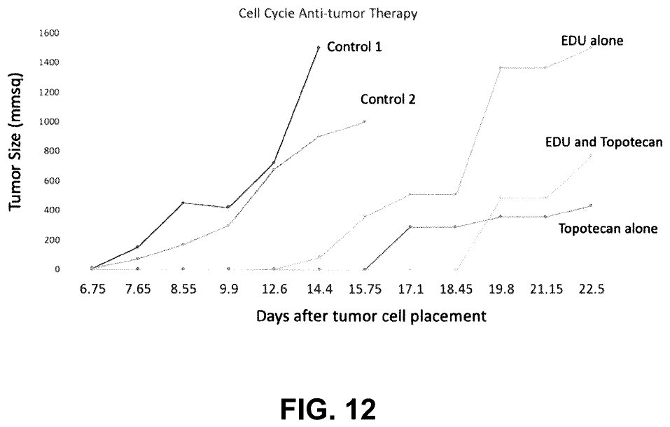

The ability of a treatment regimen to block tumor growth was determined with injections of topotecan, either alone or in combination with EDU. Interestingly, injection of EDU alone resulted in the reduction in tumor growth (FIG. 12). As with topotecan injections described above, tumor growth reduction was generally observed when drug administration was initiated 2-4 days after tumor cell inoculation, and therefore prior to the appearance of identifiable tumors. However, the results were confirmed when drug treatment was initiated after the appearance of tumors, even if such tumors had grown to 0.5 gm in size. In this case, the increase of tumor size was measured. The fact that EDU inhibits tumor growth is critical, since it is not highly toxic to animals. In general, EDU injections of 0.2 ml of 9 mg/ml drug (in phosphate buffered saline) were performed. These injections involved intermittent rest periods, generally 1-2 rest periods between 4-5 EDU injections. To increase the effectiveness of EDU injections, two such injections were at times performed within a 20 min period every 10 hours 40 min. The toxicity of these larger injections was apparent, such that the rest periods were more frequent, perhaps 2-4 rest periods every 3-6 injections (see FIG. 13A-C). In both cases, however, there was consistent inhibition of tumor growth.

More efficient tumor growth inhibition was observed with injections of topotecan, 0.07-0.25 ml; 0.2 mg/ml in phosphate buffered saline, pH 3.0. When higher volumes were injected (0.23 ml) the rest times were increased. For example, three injections at higher concentrations were often followed by 5-6 rest periods, each of 10 hours 40 minutes. With 0.2 ml injections of topotecan (0.18 ml mg/ml), three injections were often followed by 4-5 rest periods (FIGS. 12 and 13A-C). In all cases, tumor growth was dramatically inhibited. This was the case when a small tumor had already formed prior to drug treatment; or when drug treatment was initiated 2-3 days after tumor cell inoculation; and therefore 2-5 days prior to tumor appearance. Inhibition of tumor appearance was from 7-12 days longer than in untreated animals. (FIG. 12). The rate of tumor growth was also much reduced (FIG. 12). For comparison, daily cisplatin injections resulted in the delay of tumor formation of less than 3 days, and was much less effective than observed with the Cell Cycle Therapy with topotecan.

Finally, and critically, the combination of EDU and topotecan also resulted in efficient inhibition of tumor growth. Use of EDU reduced the amount of topotecan required for tumor growth reduction, but also slightly increased the overall toxicity level of topotecan (FIGS. 12 and 13A-C). EDU was always injected at 9 mg/ml (0.2 ml per injection). 1-3 such injections were followed immediately, or after 1-5 rest periods, by 1-4 injections of topotecan (0.18 ml, 0.2 mg/ml). The results obtained at present, however, are only an indication of the value of this overall approach (FIG. 12). Further study will surely reveal a treatment schedule that will optimize the effectiveness of the two drugs working together.

The use of two or more cell cycle specific drugs in combination is one of the most critical and potentially transformative aspects of this invention. In Cell Cycle Therapy, the target of any and all drugs used are cells in a given cell cycle stage. At present, only drugs toxic for S phase have been identified, but drugs toxic to mitotic cells, or cells in G1 or G2 might well be found. Each of these drugs will have the ability to kill cells in one critical cell cycle stage, and will be administered when cells are synchronized into that stage. However, any toxic compound has multiple targets. These generally vary between drugs. Accordingly, a drug toxic to S phase might also have low level toxicity to RNA synthesis, for example. A drug toxic to mitosis might also inhibit microtubule action to a small extent. Thus, if two such drugs were utilized, the inhibitor action against the primary target of the drug, cells in a given cell cycle stage, would be additive between the two. However, the side effects of each drug would be distributed between different targets, and therefore would be expected to be dramatically reduced so far as the entire individual is concerned. Therefore, the use of multiple drugs has the potential to dramatically increase toxicity to the tumor, while dramatically reducing side effects. This option is considered a central finding of this invention. Like other aspects of this invention, it is totally unique, and potentially extremely valuable.

Therefore, the optimal Cell Cycle Therapeutic treatment regimen might include multiple cell cycle specific drugs. The pattern with which these drugs would be combined together is critical. For example, one drug may be added multiple times, followed by a rest period(s) equal to multiples of the cell cycle length. Treatments with another drug would them be initiated. It would include a varying number of injections followed by a rest period(s). The protocol would be repeated. Alternatively, treatments with separate drugs could be alternated during a single treatment period. These treatment periods would then be interspersed with cell cycle-length rest periods. The treatment periods would involve any combination or sequence of administration of individual drugs. The ultimate goal is to reduce the overall rate of tumor growth and/or appearance without toxicity to the host; as with treatment schedules tested in the past (FIGS. 12 and 13A-C).

FIG. 13A-C are presented on separate sheets for size requirements. The arrow at the right of FIG. 13A could be extended to the left of FIG. 13B and the arrow at the right of FIG. 13B could be extended to the left of FIG. 13C.

### Preclinical Efficacy Study

The demonstration that topotecan and EDU are able to efficiently block tumor formation when introduced according to the timing of the cell cycle; together with the observation of cell cycle synchrony that takes place in these animals, are strong, almost convincing evidences that the Cell Cycle Therapeutic approach described above will improve cancer treatment. However, for this fact to be proven, a well-designed preclinical efficacy study is required. The one to be described here was designed to compare animals treated with the Cell Cycle Therapy approach, against the very best treatment possible not involving cell cycle considerations. Extreme care was taken to design the experiment to make this comparison valid.

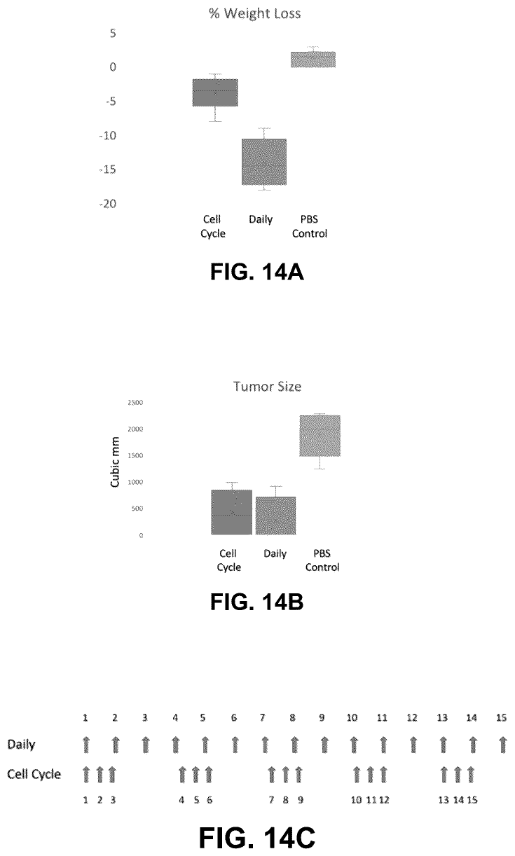

The experiment involved 24 C57Bl/6 mice, each of which received injections of 2 million Lewis lung carcinoma cells under the skin on their backs. Three days later these animals were randomly divided into three groups. The first group received three injections of topotecan separated by the length of the cell cycle (10 hours 40 min). The three-injection set was then followed by a long rest period precisely equal to 5 cell cycle lengths; and the process was repeated (FIG. 14C). For comparison, another set of mice received a single injection at the same time each day. This protocol was shown to be highly effective in blocking tumor growth, and was initially considered to be completely independent of cell cycle considerations (see above). These two protocols were perfectly matched, as they resulted in each mouse receiving 9 injections during the 9.5-day period of this experiment (or 15 injections during a 15 day time period in a longer experiment; FIG. 14C). All topotecan injections were identical (0.2 ml of 0.18 mg/ml topotecan). As a control, 8 mice received 0.2 ml injections of phosphate buffered saline (PBS) at the same time as the cell cycle group.

Recall that the overall rationale for Cell Cycle Therapy is the fact that it reduces side effects of drug treatments, allowing more drug to be added for an improved outcome. This preclinical efficacy study was designed to demonstrate this fact. Enough drug was introduced to be toxic for the animals injected once a day, without being toxic when injected according to the Cell Cycle Therapy approach. For this purpose, two critical comparisons were made in the experiment. First, the size of each tumor was determined with calipers every 1-2 days. Second, toxicity of the drug was measured by determining the weight loss of each individual animal. Both parameters at 9.5 days of treatment are presented (FIG. 14A). It is clear that there is a minimal reduction in weight for the animals injected with Cell Cycle Therapy (−3%) compared to dramatic reduction in weight for animals with daily injections (−15%, FIG. 14A). At 9.5 days this particular experiment was terminated due to the weight loss resulting from the high level of toxicity of the daily injection group. The animals in the Cell Cycle Therapy group, on the other hand, received injections for another week without developing significant toxicity.

This result substantiates the observations made above, that injections separated by the length of the cell cycle have reduced toxicity. The validity of Cell Cycle Therapy, however, is established by the fact that despite reduced toxicity, the injections separated by cell cycle length retained almost equal ability to block tumor growth compared to daily injections (FIG. 14B). Thus, the average tumor size of animals treated with topotecan by either protocol was ¼th the size of the PBS treated animals. All these results were highly significant statistically.

### Combined EDU and Topotecan Treatments:

As emphasized above, one potentially powerful aspect of Cell Cycle Therapy is the opportunity to use different drugs, each targeting cells in a single cell cycle phase, but each of which would presumably have different off-target side effects. In this way, the toxicity to out of cycle tumor cells would be maximized, while the side effects of these drugs to normal tissues would be distributed among these different tissues, and therefore would be more tolerable for the animal. The effectiveness of a treatment including both EDU and topotecan is illustrated in FIG. 12 and FIG. 13A-C. Additionally, the power of dual treatment was revealed in experimental treatments during the formative stages of this work, when it was observed that a particular combination therapy actually reduced tumor size. Topotecan alone has never been found in these studies to reduce tumor size, regardless of the treatment protocol. Experiments were, therefore, designed to demonstrate that a protocol involving both EDU and topotecan could be more effective in blocking tumor growth than treatment with topotecan alone.

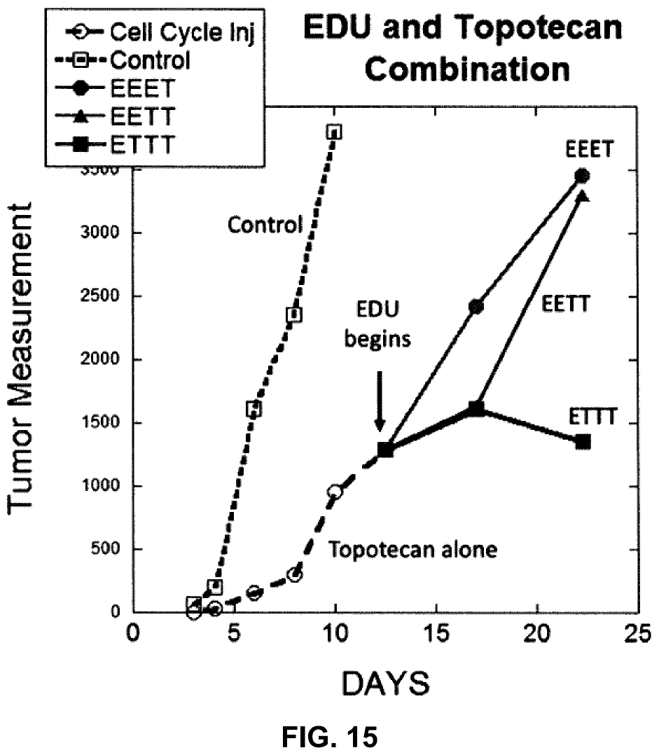

The experiment to establish the utility of dual treatment involved two steps. The first step was similar to the above experiment (FIG. 14A-C), where Lewis lung carcinoma cells were introduced into mice, which were then treated with three topotecan injections followed by five rest periods. It is critical to use this protocol first as a means to determine the tumor growth inhibition efficiency of topotecan alone. When the tumors treated in this way had grown to approximately 4% of body weight, the treatment protocol was changed to include EDU (9 mg/ml). Thus, growth inhibition by topotecan alone could be compared to inhibition with the combination therapy in the same animals. Three groups of two animals each were treated with separate protocols. In the first, EDU injections were repeated three times, followed by one topotecan injection. In the second protocol, two EDU injections were followed by two topotecan injections. In the third group, one EDU injection was followed by three topotecan injections (FIG. 15). These 4 injections were followed by a rest period equal to five cell cycles as in the past.

None of the dual injected animals experienced toxicity as indicated by weight loss, even when the experiment was extended for an additional two weeks. Only the group receiving one EDU and three topotecan injections, however, displayed reduced tumor growth. Significantly, the animals thus treated not only remained vigorous, but their tumors nearly stopped growing after the second treatment cycle. Comparison of the rate of tumor growth with topotecan alone can easily be contrasted to the near blockage of tumor growth when EDU was added to treatment protocol of these same tumors. This result is a clear example of the extraordinary potential of Cell Cycle Therapy. It should be emphasized that the results reported so far will only improve as more cell cycle specific drugs are identified, and when the optimal protocols to utilize them are identified. As remarkable and unprecedented as the results reported here are, they should be considered only a beginning for Cell Cycle Therapy.

These preclinical efficacy studies show that Cell Cycle Therapy is a clear improvement over even the most effective traditional therapy. Traditional, daily injections of topotecan at the concentration selected results in a highly effective inhibition of Lewis lung carcinoma tumors. This anti-tumor effect is of little use, however, because the animals themselves are poisoned by this level of topotecan. Only when the injections are given according to Cell Cycle Therapy is the full effectiveness of tumor growth inhibition made available in a non-toxic protocol. Inclusion of a second cell cycle drug, such as EDU results in a dramatic increase in effectiveness.

This written description uses examples to describe the disclosure, including the best mode, and also to enable any person skilled in the art to make and use the disclosure. Other examples that occur to those skilled in the art are intended to be within the scope of the present disclosure if they have structural elements that do not differ from the same concept, or if they include equivalent structural elements with insubstantial differences. It will be appreciated that variants of the above-disclosed and other features and functions, or alternatives thereof, may be combined into many other different systems or applications. Various presently unforeseen or unanticipated alternatives, modifications, variations or improvements therein may be subsequently made by those skilled in the art which are also intended to be encompassed by the following claims.

The following references provide background information and are incorporated by reference herein in their entireties.

### REFERENCES

ADDIN Mendeley Bibliography CSL_BIBLIOGRAPHY Clevers, H. and Batlle, E. (2013) ‘SnapShot: The intestinal crypt’, Cell, 152(5), pp. 1198-1198.e2. doi: 10.1016/j.cell.2013.02.030.
Gangwar, R. S., Gudjonsson, J. E. and Ward, N. L. (2022) ‘Mouse Models of Psoriasis: A Comprehensive Review’, Journal of Investigative Dermatology, 142(3), pp. 884-897. doi: 10.1016/j.jid.2021.06.019.
Hitomi, M. and Stacey, D. W. (2001) ‘Ras-dependent cell cycle commitment during G2 phase’, FEBS Letters. doi: 10.1016/80014-5793(01)02115-9.
Van Moorsel, C. J. A. et al. (1999) ‘Scheduling of gemcitabine and cisplatin in Lewis Lung tumour bearing mice’, European Journal of Cancer, 35(5), pp. 808-814. doi: 10.1016/S0959-8049(99)00004-0.
Smith, M. R., Degudicibus, S. J. and Stacey, D. W. (1986) ‘Requirement for c-ras proteins during viral oncogene transformation’, Nature. doi: 10.1038/320540a0.
Stacey, D. W. (2010) ‘Three observations that have changed our understanding of cyclin D1 and p27 Kip1 in cell cycle control’, Genes and Cancer. doi: 10.1177/1947601911403475.
Stacey, D. W. and Allfrey, V. G. (1976) ‘Microinjection studies of duck globin messenger RNA translation in human and avian cells’, Cell. doi: 10.1016/0092-8674(76)90136-7.

1. A process of synchronizing normal cells comprising:
administering a plurality of doses of a camptothecin analogue or a pharmaceutically acceptable salt thereof; wherein each successive dose is administered from about 9 hours and 45 minutes to about 10 hours and 45 minutes after the previous dose.

2. The process of claim 1, wherein the camptothecin analogue is topotecan.

3. The process of claim 1, wherein the camptothecin analogue is irinotecan.

4. The process of claim 1, wherein the plurality of doses comprises at least 4 doses.

5. The process of claim 1, wherein the plurality of doses comprises at least 6 doses.

6. The process of claim 1, wherein the plurality of doses comprises at least 7 doses.

7. The process of claim 1, wherein the plurality of doses comprises at most 12 doses.

8. The process of claim 1, wherein the plurality of doses is between 4 and 6, with a rest period of 40 to 70 hours; after which the dosing is repeated for an extended period of time.

9. The process of claim 1, wherein each dose comprises 0.5 μg/kg to about 1.5 μg/kg.

10. The process of claim 1, wherein each dose comprises 0.3 μg/kg to about 3 μg/kg.

11. The process of claim 1, wherein the total amount of topotecan administered is at most about 100 μg/kg.

12. The process of claim 1, wherein the administration is via injections.

13. The process of claim 1, wherein the process does not result in the synchronization of tumor cells; or any type of unwanted cell type with altered growth properties compared to the normal tissue to be preserved.

14. The process of claim 1, wherein the process does result in the synchronization of tumor cells; or any type of unwanted cell type with altered growth properties compared to the normal tissue to be preserved. In this case, additional treatment with a targeted growth regulatory drug will be administered to temporarily block cell cycle progression of the normal but not the unwanted (tumor) cells. During this temporary blockage treatment with a potent proliferative toxin will kill the unwanted (tumor) cells with altered growth properties, but not the normal cells blocked at that time in cell cycle progression. Finally, all drugs will be withdrawn. The normal cells will have survived and will continue to proliferate normally, while the unwanted (tumor) cells will have been poisoned.

15. A process for treating small intestine cancer comprising: administering a plurality of doses of topotecan or a pharmaceutically acceptable salt thereof; wherein each successive dose is administered from about 9 hours and 45 minutes to about 10 hours and 45 minutes after the previous dose.

16. The process of claim 15, wherein the plurality of doses comprises at least 4 doses.

17. The process of claim 15, wherein the plurality of doses comprises at least 6 doses.

18. The process of claim 15, wherein the plurality of doses comprises at least 7 doses.

19. The process of claim 15, wherein each dose comprises 0.5 μg/kg to about 1.5 μg/kg.

20. The process of claim 15, wherein each dose comprises 0.7 μg/kg to about 1.5 μg/kg.
